(19) United States
(12) Patent Application Publication
(10) Pub. No.: US 2016/0146841 A1
(43) Pub. Date: May 26, 2016

(54) METHOD FOR DETERMINING FREE COPPER

(71) Applicant: CANOX4DRUG S.P.A., Capurso BA (IT)

(72) Inventors: Nicola Antonio COLABUFO, Triggiano (IT); Francesco BERARDI, Noicattaro (IT); Marcello LEOPOLDO, Bari (IT); Roberto PERRONE, Bari (IT); Rosanna SQUITTI, Roma (IT)

(21) Appl. No.: 14/888,088

(22) PCT Filed: Apr. 29, 2014
(86) PCT No.: PCT/IB2014/061079

§ 371 Date: Oct. 29, 2015

(30) Foreign Application Priority Data
Apr. 29, 2013 (IT) RM2013A000253

(51) Int. Cl. G01N 33/84; G01N 33/52; G01N 31/22

(52) U.S. Cl. CPC G01N 33/84; G01N 31/22; G01N 33/52; G01N 2800/2821

(57) ABSTRACT

The present invention relates to a new method for the determination of ‘free’ copper concentration in serum, i.e. the portion of serum copper not structurally bound to ceruloplasmin. The present invention also refers to a method with a high degree of sensitivity and precision for the determination of free copper in serum samples of patients with Alzheimer's disease.

The present invention relates to a new method for the determination of ‘free’ copper concentration in serum, i.e. the portion of serum copper not structurally bound to ceruloplasmin. The present invention also refers to a method with a high degree of sensitivity and accuracy for the determination of free copper in serum samples of patients with Alzheimer's disease.

### STATE OF THE PRIOR ART

The determination of serum copper is of primary importance in a large number of diseases as for example in the Alzheimer's disease (AD). Alzheimer's disease is a neurological disorder characterized by memory loss and progressive dementia. The cause of the disease appears closely related to the aggregation within the brain of the beta-amyloid (Aβ) protein and tau peptides. Moreover, the epsilon 4 allele of the apolipoprotein E (APOE) gene has been proven to increase Alzheimer's Disease risk. On the ‘amyloid cascade’, which is recognized as the most popular hypothesis for Alzheimer's disease onset, new details have recently emerged. In fact, diverse pathogenic pathways have been postulated to contribute to Alzheimer's disease onset and progression. There is abundant evidence proving that oxidative stress, mainly via metal redox reactions, can cause brain damage to the Alzheimer's Disease brain. Specifically, it has been proposed that the hyper-metallization of the beta-amyloid protein can be at the basis of redox cycles of oxidative stress and H₂O₂ production, determining Aβ protein oligomer formation and precipitation. A derangement of metal homeostasis leads to formation of free copper that may feed the brain copper reservoir and enter Aβ-oxidative stress cycles, generating pleiotropic effects on the Alzheimer's Disease. This thesis is now supported by several lines of evidence showing that free copper is slightly but significantly increased in the serum of Alzheimer's disease patients.

Ceruloplasmin is the major copper-carrying protein in the blood, and it binds structurally 6 atoms of copper to form an active form of the protein, which can account for about 85-95% of circulating copper, the remaining copper being defined as free. In previous studies the inventors used to determine free copper in serum starting from copper and ceruloplasmin measures, with the calculation as follows: serum copper concentrations were double-checked by measuring them either with the atomic absorption spectroscopy technique utilizing an A Aanalyst 300 Perkin Elmer atomic absorption spectrophotometer equipped with a graphite furnace with platform HGA 800, or according to the colorimetric method of Abe et al. Clin Chem 1989 (Randox Laboratories, Crumlin, UK); ceruloplasmin concentration was analyzed by immunoturbidimetry assay (Horiba ABX, Montpellier, France) according to Wolf P L Crit Rev Clin Lab Sci 1982, for each serum copper and ceruloplasmin pair it has been computed the amount of copper bound to ceruloplasmin (CB) and the amount of copper not bound to ceruloplasmin (‘free’ copper) following standard procedures described in Walsh et al. Ann Clin Biochem 2003. This calculation expresses ‘free’ copper in μmol/L and is based on the evidence that ceruloplasmin contains 0.3% copper. Moreover, the inventors have recently described a procedure for measuring ceruloplasmin oxidase activity which uses o-diansidine dihydrochloride as a substrate.

Previously, methods for determining ceruloplasmin amount starting from the protein's oxidase activity with a commercial standard (Human Serum Ceruloplasmin, Sigma-Aldrich) have been described, but spectroscopic analysis revealed a decay in the protein peak of absorbance, decreasing the confidence in using the enzymatic detection to quantify the protein amount, necessary to estimate the free copper value.

Quantification of copper and ceruloplasmin based on the enzymatic methods described in the state of the art entails several drawbacks, such as, e.g., a high cost, the variable purity of commercially available ceruloplasmin, the general recommendation to report serum enzymes in International Units (UI) and a low degree of accuracy of the determined concentration.

Hyo Jung Sung et al. (J. Am. Chem. Soc. 2009) describes the synthesis and the use of coumarin probes for the determination of free copper in biological systems.

Scope of the present invention is to provide new methods and kits for measuring free copper in serum which do not entail the drawbacks of the prior art.

### SUMMARY OF THE INVENTION

Object of the present invention is an in vitro method for determining the concentration of free copper in a serum sample comprising the following steps:

a) loading said serum sample on a resin for solid phase extraction obtaining a bonded fraction, and an eluted fraction comprising free copper;

b) determining the concentration of free copper in the fraction eluted in step a) using a coumarin fluorescent probe.

A further object of the invention is an in vitro method for determining the concentration of free copper for the diagnosis of Alzheimer's disease in a patient comprising the same steps a), b) and a further step c) of comparing the value determined in step b) with a threshold value (cut-off), wherein a higher concentration of free copper confirms the clinical diagnosis of Alzheimer's disease.

A further object of the invention is an in vitro method for determining the concentration of free copper for the prognosis of Alzheimer's disease in a patient in which the steps a) and b) of the method are repeated on serum samples collected from said patient at subsequent time-points and the progression in time of the concentration of free copper in these samples is evaluated.

A further object of the invention is an in vitro method for determining the concentration of free copper for the evaluation of the predisposition to conversion from a state of mild cognitive impairment (MCI) to Alzheimer's disease in a patient suffering from mild cognitive impairment comprising the same steps a) and b) and a further step c) of comparing the value determined in step b) with a threshold value (cut-off), wherein a higher concentration of free copper points out the conversion from mild cognitive impairment to Alzheimer's disease.

A further object of the invention is a kit for the detection of free copper in serum comprising one or more devices for chromatographic extraction on a solid phase and one or more coumarin fluorescent probes.

The inventors have observed that free copper concentration in serum is inaccurately estimated due to the presence of blood proteins; moreover, they have also observed that various methods of separating low-molecular weight chemical elements from blood proteins, e.g. with membrane filtering devices, do not enable to accurately determine the concentration of free copper. The invention described herein is based on the selection of a step of separating the free copper from blood proteins and on the selection of a specific class of fluorescent probes.

The method of the present invention entails several advantages compared to the determination methods of the state of the art:
enables to determine the concentration of free copper in serum with a high precision and accuracy;
allows to determine the concentration of free copper in serum with very reduced costs and times;
it is an easily automatable method.

### BRIEF DESCRIPTION OF THE FIGURES

FIG. 1. Calibration curve of the coumarin fluorescent probe 7-(Diethylamino)-2-oxo-N-((pyridin-2-yl)methyl)-2H-chromene-3-carboxamide in the presence of Cu⁺⁺(10⁻⁴ M) in HEPES: DMSO 9:1 (λex=430 nm, 490 nm λem).

FIG. 2. Calibration curve of the coumarinic fluorescent probe 7-(Diethylamino)-2-oxo-N-((pyridin-2-yl)methyl)-2H-chromene-3-carboxamide in the presence of Cu⁺⁺(10⁻⁵ M) in HEPES: DMSO 9:1 (λex=430 nm, 490 nm λem).

FIG. 3. Receiver operating characteristic (ROC) curve. 702 samples have been analyzed according to one embodiment of the present invention. The curve shows that using the present invention a diagnosis of Alzheimer's disease can be obtained with high specificity (80%) and discrete sensitivity (60%).

FIG. 4. Model to predict the probability (Mini-Mental State Examination) of worsening in patients affected by Alzheimer's disease according to free serum copper levels. Circles represent the value of free serum copper of the patients. The line represents the model of the predicted probability of Mini-mental State Examination worsening. Those patients from the current study panel who had a z-score higher than −0.138, corresponding to a free copper value of 2.1 μmol/L, had an increased probability to worsen than those patients who had their ‘free’ copper values below such levels.

FIG. 5. Free copper concentration can also be used to predict the percentage of subjects complaining mild cognitive impairment, who will develop Alzheimer's Disease. Mild cognitive impairment subjects with a free copper concentration of >1.6 μM have a higher percentage of conversion to Alzheimer's Disease.

FIG. 6. A photo of an apparatus for carrying out the method of the present invention according to one embodiment.

FIG. 7. Spectrophotometric analysis of a serum sample separated by extraction on a solid phase. Spectrophotometric analysis enables to verify protein presence in the filtrate; the higher the protein presence, the worse is the performance in terms of free copper recovery, as these proteins mask copper reading. As the curve reduces in width, protein composition decreases and therefore free copper recovery improves.

FIG. 8A and 8B. Spectrophotometric analysis of a serum sample separated by membrane filtration.

FIG. 9. Polynomial and “non-parametric-lowess” (locally weighted scatterplot smoothing) linear regression analyses are depicted, obtained with values of copper not bound to ceruloplasmin (non-cp copper) determined by the reference test (calculated copper) of the state of the art or by the method according to the present invention (C4D).

FIG. 10. (95%) confidence intervals of non-cp copper in healthy subjects, in mild cognitive impairment (MCI) subjects and in Alzheimer's Disease (AD) subjects determined by the reference test of the state of the art (calculated copper) or by the method according to the present invention (C4D) are depicted.

FIG. 11. ROC curves, obtained by using values of non-cp copper concentration determined according to the reference test (calculated copper) of the state of the art or by the method according to the present invention (C4D) are depicted.

### DETAILED DESCRIPTION OF THE INVENTION

As previously indicated, the present invention relates to an in vitro method for the determination of the concentration of the free copper in a serum sample. In the present description the term “free copper” means copper in general circulation which is not structurally bound to ceruloplasmin. It is also recently named ‘labile’ copper, referring to its properties of being labile bound to albumin, small peptides, amino acids and other micro-nutrients, and of being easily exchangeable among them. Free copper is a small molecular weight copper which can easily reach brain tissues, crossing the blood-brain barrier.

In order to separate free copper from the blood proteins of a serum sample, the method comprises a first step of (a) solid phase extraction (SPE) chromatography. The serum sample could be obtained from whole blood according to the procedures known to the technician in the field, e.g. by centrifuging. The serum before being subjected to separation could be properly diluted, preferably according to a dilution factor between 1 and 10. The serum could, e.g., be diluted in physiological solution (0.9% NaCl) which could also be used as mobile phase in chromatography.

The serum sample is loaded (seeded) on a solid phase (a resin able to bind blood proteins), generally in small chromatography columns, e.g., 200 mg, 300 mg, 400 mg, 500 mg, 600 mg ones. Blood proteins present in the serum sample, ceruloplasmin included, are adsorbed on the solid phase, whereas the fraction eluted from solid phase, comprising copper, is collected and subjected to the second step b) of the method. In the present description, therefore, by ‘eluted fraction’ it is meant the fraction not adsorbed on the resin used in the solid phase extraction chromatography (chromatographic extraction on a solid phase).

The sample could be loaded on the solid phase by a peristaltic pump with a flow rate between, e.g., 100 μl/min and 1 ml/ml, for instance 200, 300, 400, 500 μl/min.

In step a) a polyolefin, preferably a thermoplastic polyolefin selected, e.g., from polyethylene (PE), polypropylene (PP), polymethylpentene (PMP), polybutene-1 (PB-19) could be used as solid phase. Said solid phase could have, e.g., a degree of crystallinity between 35 and 75%.

According to one embodiment of the invention, as solid phase a resin of ultra-high molecular weight polyethylene (i.e. with an atomic mass between 3 and 6 MDa) will be used, for instance commercially available from Sigma-Aldrich with cat. # 434264-1KG (Ultra-high molecular weight polyethylene (UHMPE) and any other equivalent commercial resin). The entire step a) is therefore extremely quick and easily automatable; moreover, the solid phase, once regenerated with a suitable solvent, like e.g. methanol, could be reused for other separations with economic advantages.

The method comprises a second step of (b) determining the copper in the fraction eluted in step a) using a coumarin fluorescent probe. Coumarin fluorescent probes are chelating fluorescent probes for which a decay in fluorescence emissions could be recorded when it binds [Cu⁺⁺]. The coumarin fluorescent probes may be selected for example from compounds having the following general structural formula:

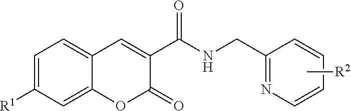

wherein

R¹ is N[(CH₂)nCH₃]₂ with n from 0 to 5;

R² is H, F, CI, Br, NO₂, OCH₃, cyclohexyl.

According to one embodiment of the present invention said coumarin fluorescent probe is selected from the group above wherein R¹ is N[(CH₂)nCH₃]₂ with n=1 and R² is H, F, Cl, Br, NO₂, OCH₃, cyclohexyl either in ortho-, para- or meta-position.

According to another embodiment of the present invention said coumarin fluorescent probe is selected from the group above wherein R¹ is N[(CH₂)nCH₃]₂ with n=2 and R² is H, F, Cl, Br, NO₂, OCH₃, cyclohexyl either in ortho-, para- or meta-position.

According to another embodiment of the present invention said coumarin fluorescent probe is selected from the group above wherein R¹ is N[(CH₂)nCH₃]₂ with n=3 and R² is H, F, Cl, Br, NO₂, OCH₃, cyclohexyl either in ortho-, para- or meta-position.

According to another embodiment of the present invention said coumarin fluorescent probe is selected from the group above wherein R¹ is N[(CH₂)nCH₃]₂ with n=4 and R² is H, F, Cl, Br, NO₂, OCH₃, cyclohexyl either in ortho-, para- or meta-position.

According to another embodiment of the present invention said coumarin fluorescent probe is selected from the group above wherein R¹ is N[(CH₂)nCH₃]₂with n=5 and R² is H, F, Cl, Br, NO₂, OCH₃, cyclohexyl either in ortho-, para- or meta-position.

According to another embodiment of the present invention said coumarin fluorescent probe is 7-(Diethylamino)-2-oxo-N-((pyridin-2yl)methyl)-2H-chromene-3-carboxamide having the following structural formula:

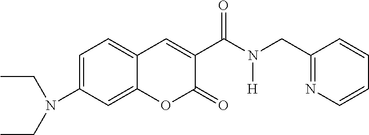 [SMILES: CCN(CC)c1ccc2cc(C(=O)NCc3ccccn3)c(=O)oc2c1]

The coumarin fluorescent probe may be used for example in organic solvents as EtOH, MeOH, DMSO mixed to buffer solutions as PBS or Hepes. In one embodiment the coumarin fluorescent probe is used in a solution of HEPES:DMSO.

The coumarin fluorescent probes will be used in the reaction with the sample preferably in a concentration range between 0.1 and 10 μM, for example 1, 2.5, 5.0, 91 μM. The inventors found that in this range there is a direct correlation between the concentration of free copper and the fluorescence emission, the excitation wavelength (λex) is for example 430 nm and the adsorption wavelength (λem) 490 nm.

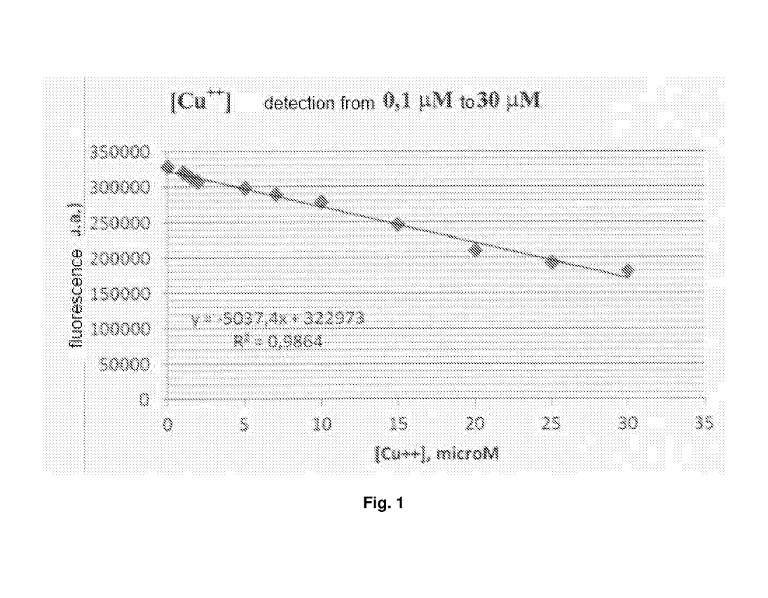

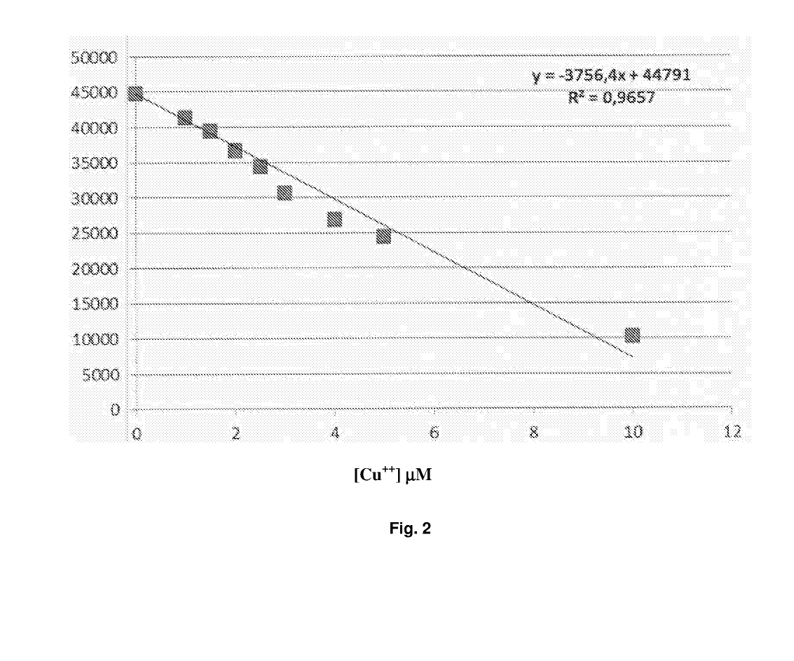

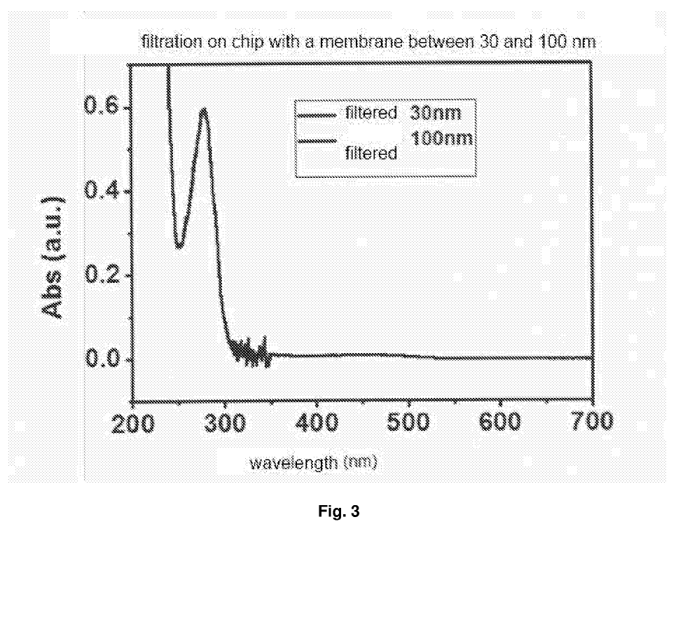

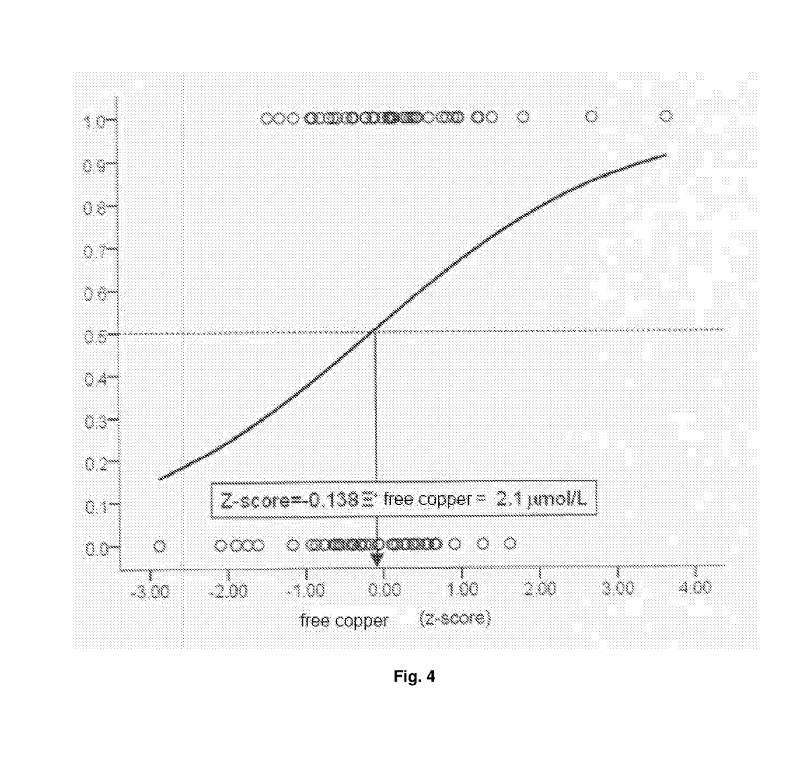

In order to determine the concentration of the free copper, step b) may comprise a further step of preparing a calibration curve. To prepare the calibration curve, plural aliquots with a known concentration of copper may be used. Preferably this curve will be in the range between 0.1 and 10 μM (see FIG. 1).

As previously reported in patients affected by Alzheimer's Disease, serum copper not bound to ceruloplasmin (‘free’ copper) appears elevated and the increase, though slight, is normally sufficient to distinguish Alzheimer's Disease patients from healthy elderly subjects (also in the early stages of the disease).

Hence it is an object of the present invention an in vitro method for the diagnosis of Alzheimer's disease in a patient suspected of having Alzheimer's Disease comprising a further step c) of comparing the value determined in step b) with a threshold value (cut-off), wherein a higher concentration of free copper confirms the clinical diagnosis of Alzheimer's disease.

By the expression “in vitro method for the diagnosis of Alzheimer's disease” it is meant a method for confirming the clinical diagnosis of Alzheimer's Disease in a patient suspected of having Alzheimer's Disease.

Evidently, if before being loaded on the chromatography the serum has been diluted according to a certain dilution factor, in step c), of comparing with the threshold value, the free copper concentration determined in step b) will have to be multiplied by the dilution factor.

The threshold value (cut-off) of copper may be determined for example by means of ROC (Receiver Operating Characteristic) curves obtained by processing the concentrations of a set of samples (statistically significant) of healthy individuals and individuals with Alzheimer's disease. Through such processing were obtained threshold values between 0.5 and 50 μm, preferably between 0.5 and 3 μm, for example 1, 1.5, 2, 2.5, 3 μm.

Preferably said diagnosis method will be used as a confirming test for a clinical diagnosis of Alzheimer's disease in a patient suspected of having Alzheimer's Disease with a ‘copper phenotype dysfunction’.

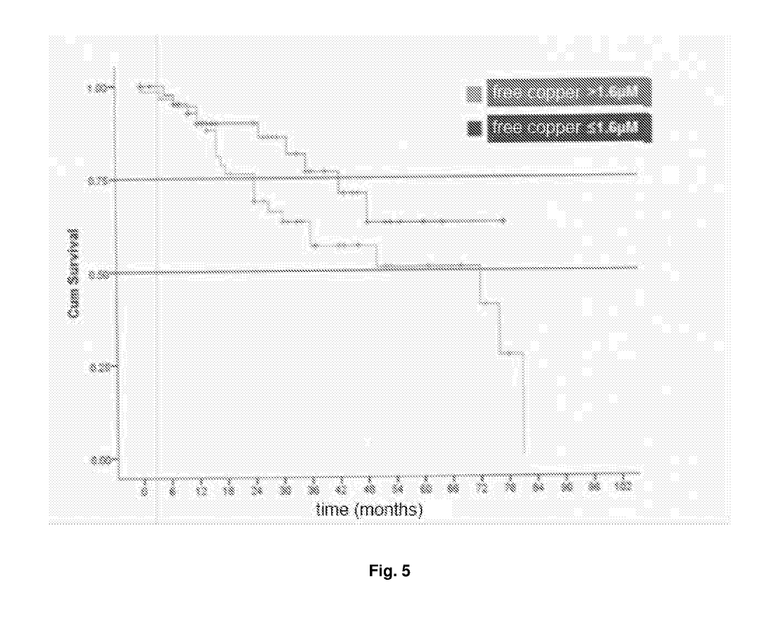

As shown by Squitti et al., Neurology (2009) to monitor the prognosis of Alzheimer's Disease in a patient as well as to predict the conversion from mild cognitive impairment (Mild cognitive Impairment) to Alzheimer's disease it is important to determine the concentration of free copper in the serum of said patient (FIG. 5).

The clinical condition of Mild cognitive impairment is characterized by memory impairments, verifiable via objective measures, not yet granting the definition of dementia. The importance of an accurate diagnosis lies in the fact that, despite the mildness of the condition, Mild cognitive impairment is normally considered as a precursor of Alzheimer's disease. This is due to the high statistical rate of progression from Mild cognitive impairment to Alzheimer's Disease.

Normally, the annual conversion rate from a healthy condition to Alzheimer's disease ranges from 0.17% to 3.86%. The conversion rate from mild cognitive impairment to Alzheimer's disease is remarkably higher, ranging from 6% to 40%. In some cases, Mild cognitive impairment can be a benign condition, with no progression into dementia. Free copper concentration discriminates Mild cognitive impairment subjects from healthy control individuals, as revealed by comparing the means of the two groups (FIG. 5). Free copper concentration can also be used to predict the percentage of subjects with mild cognitive impairment, who will develop Alzheimer's Disease. Mild cognitive impairment subjects with free copper concentration >1.6 μM have a higher percentage of conversion to Alzheimer's disease, that is 17% per year, with respect to those mild cognitive impairment subjects with copper ≦1.6 μM, that is 10% per year. Kaplan-Meier statistical analysis confirms that mild cognitive impairment subjects with copper >1.6 μM have a higher rate of conversion to Alzheimer's Disease than those with copper ≦1.6 μM, their percentage of conversion to Alzheimer's Disease being between 24-35% within the first two years, compared to 25-30% of those mild cognitive impairment subjects with free copper ≦1.6 μM who convert within 3 years and a half. Limiting the analysis to the five-year follow-up, the percentage of conversion to Alzheimer's disease in the Mild cognitive impairment subjects with copper ≦1.6 μM is less than 50%, while in the mild cognitive impairment cohort with copper >1.6 μM 50% of the patient convert within 4-6 years (FIG. 5).

In one embodiment the method of the present invention is used for predicting the conversion from a state of mild cognitive impairment (MCI) to Alzheimer's disease in a patient suffering from mild cognitive impairment comprising a step c) of comparing the value determined in step b) with a threshold value (cut-off), in which a higher concentration of copper indicates the conversion from Mild Cognitive Impairment to Alzheimer's disease. This threshold value is for example between 0.5 and 3 μM, preferably 1.6 μM. Steps a) and b) of said prediction method may be performed according to any embodiments of the above-disclosed steps a) and b).

A further object of the present invention is an in vitro method for the prognosis of Alzheimer's disease in a patient wherein the steps a) and b) of the method according to any embodiments of the above-disclosed steps a) and b) are carried out on more samples of said patient collected in different moments and the quantification of data obtained from each sample are compared one to the other, thus constructing a progression in time of the concentration of free copper in the serum samples of said patient.

A further object of the present invention is a kit for the detection of free copper in serum comprising means and instructions for performing a chromatographic extraction on a solid phase and one or more fluorescent coumarin probes. The means for performing a chromatographic extraction on a solid phase are, for instance, chromatography columns containing solid-phase resin. In one embodiment said means comprise as solid phase ultra-high molecular weight polyethylene. In a further embodiment said coumarin fluorescent probe is selected from the compounds having the structural formulas described above.

In one embodiment the kit further comprises one or more aliquots of controls having a known titer of copper; these controls may be used to prepare a calibration curve.

Examples aimed at illustrating some embodiments of the present invention are reported here below; in no way such examples are to be construed as a limitation of the present description and of the subsequent claims.

### EXAMPLES

### Example 1

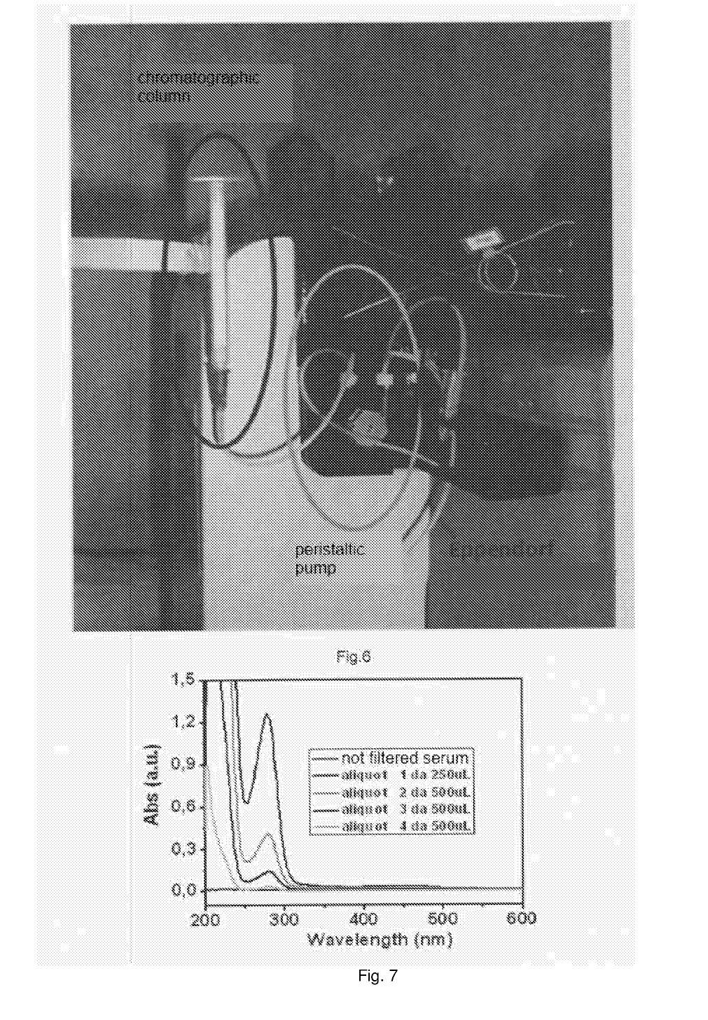

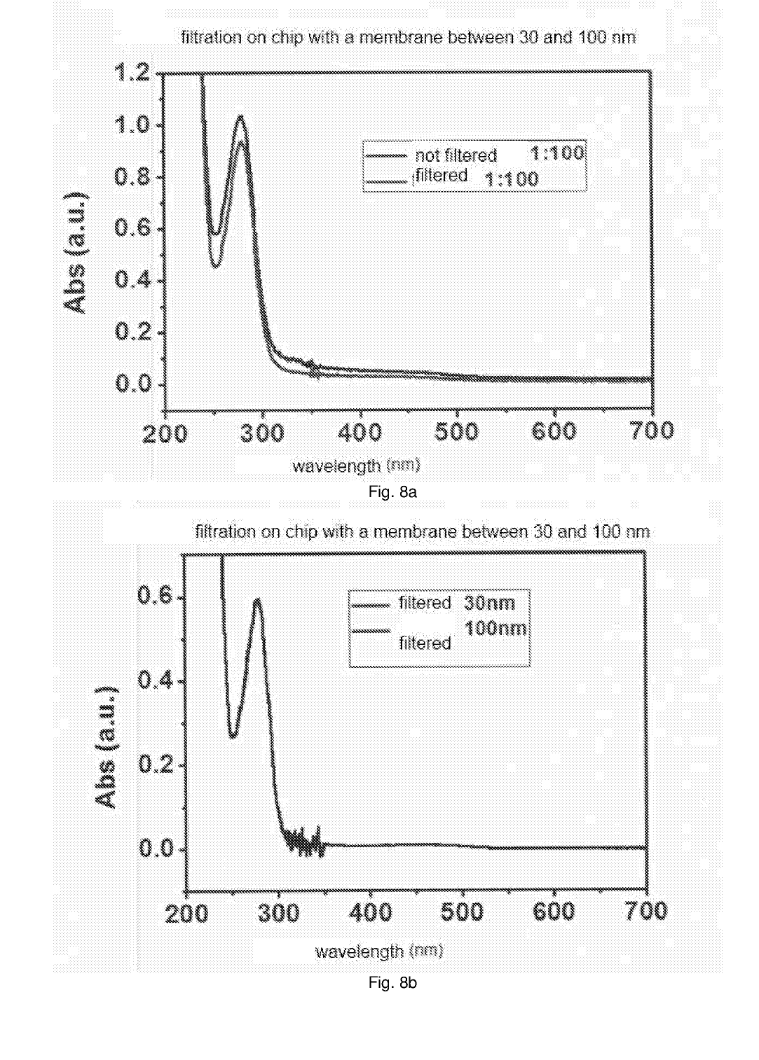

For blood protein separation, the solid phase extraction (SPE) chromatography method was set up. As solid phase, ultra-high molecular weight polyethylene (UHMPE) resin (Sigma-Aldrich cat. # 434264-1KG) was used, capable of interacting and retaining serum proteins. As mobile phase, in order to prevent the release of protein (ceruloplasmin)-bonded copper, rather than pure water physiological solution (0.9% NaCl) was used, sucked by a peristaltic pump to maintain a constant elution flow (flow rate: 400 μl/min). 1-ml chromatography columns were packed with 500 mg of resin (FIG. 6) and conditioned by using two different strategies:
500 mg of resin, put in a column, were conditioned with 6 ml of physiological solution.
500 mg of resin were suspended in about 3 ml of methanol, then used to load the column. Then, 6 ml of distilled water were eluted through the column to completely remove methanol, followed by 6 ml of physiological solution. Then, in both cases, 50 μl of serum were loaded and eluted with physiological solution. The first 250 μl of eluate and subsequent 500 μl aliquots were separately collected. Spectrophotometric analysis has detected protein absence in the 250 μl aliquots (aliquot 1 of FIG. 7), whereas protein presence is observed in the subsequent 500 μl aliquots (aliquot 2 and 3 of FIG. 7). For any laboratory needs, in order to abate times for collection of the aliquots of interest, it is possible to improve the protocol by reducing the mobile phase volumes needed for column conditioning. One advantage of said technique is given by the possibility of regenerating the columns by eluting the methanol-adsorbed proteins (about 2 ml). Columns regenerated and used for 3 subsequent separations confirmed the expected results: spectrophotometric analysis detects protein absence in 250 μl aliquots. The entire protocol develops in a maximum of 30 minutes. The optimized method decreases times to 20 or 15 or 10 minutes, down to 6 cycles/hour.

### 1. Comparative Experiments

Free copper concentration was determined in various serum samples with known free copper concentrations. The list of samples analyzed and of their concentration is reported in Table 1. Free copper concentration in the samples was determined by the method of the present invention, in particular according to the embodiment described in detail in Example 1 and in parallel, by using in the separating step filtration membranes instead of the chromatographic extraction on a solid phase (SPE).

The results obtained indicate that by using different types of filtration membranes, however, a reduction of 35 to 77% is had in the recovery of free copper contained in the sample. In particular, the experiments indicate that membrane devices do not allow to remove proteins from serum samples diluted 1:10 (maximum dilution allowed for a Cu assay).

On the contrary, the filtration yield using chromatographic extraction on a solid phase (SPE) is proportional to the amount of serum seeded. Moreover, in the MeOH eluate a protein amount is obtained that is approximately inversely proportional to the filtered amount, to confirm the accuracy of the method (the proteins retained after 10 μl filtration in 1 mL are >25 μl in 2.5 mL >50 μl in 5 mL).

The protein fraction, seeding 50 μl, is collected in the first two 500 μl fractions. Then, all serum is collected in 1 mL. Even excluding an initial 250 μl fraction, the other two fractions are those containing proteins.

To sum up, filtration with membrane devices is not efficient, whereas filtration with chromatographic extraction on a solid phase is more accurate and quicker.

TABLE 1
Patients and control sera selected in a clinical setting by the
Department of Neuroscience, Fatebenefratelli Hospital, Rome

| ID | Micromolar concentration | classification |
|---|---|---|
| 1647 | 0.1 | control |
| 1650 | 0.2 | control |
| 1665 | 0.1 | control |
| 1666 | 0.2 | control |
| 1667 | 0.5 | control |
| 1780 | 3.2 | Alzheimer |
| 1794 | 3.8 | Alzheimer |
| 1796 | 4.5 | Alzheimer |
| 1799 | 3.55 | Alzheimer |
| 1802 | 6.2 | Alzheimer |
| 1818 | 3.3 | Alzheimer |
| 1839 | 5.6 | Alzheimer |
| 1848 | 3.3 | Alzheimer |
| 1855 | 3.8 | Alzheimer |
| 1856 | 1.6 | Alzheimer |
| 1876 | 2.2 | Alzheimer |
| 1890 | 2.8 | Alzheimer |
| 1899 | 4.2 | Alzheimer |
| 1901 | 0.8 | control |
| 1926 | 2.0 | Alzheimer |

### 2. Comparative Experiments

In the experiments described below, the concentration of “free copper” in sera obtained from a significant sample of individuals was determined, both with the method according to the present invention and with the reference method (computed copper) used in the state of the art and described in Walsh et al. Ann Clin Biochem 2003.

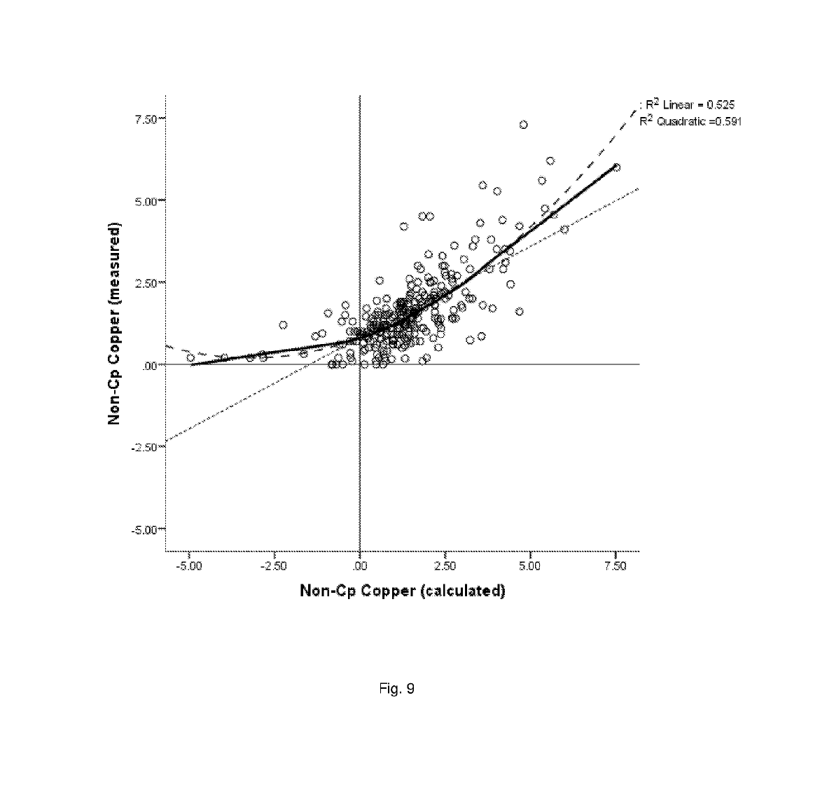

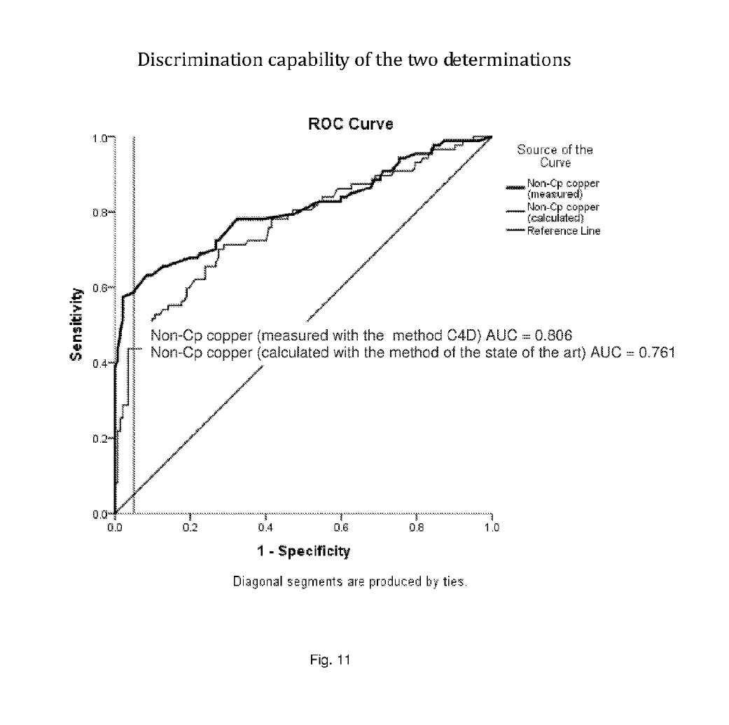

The method according to the present invention is more shortly denoted hereinafter and in FIGS. 9-11 also by the name C4D (acronym of Canox4Drug).

The following analyses are reported:

a. Comparison with the reference test of the state of the art;

b. C4D test precision;

c. C4D test linearity;

d. C4D test detection limits;

e. C4D test reference interval

f. Discriminant validity

i. Comparison of means;

ii. Diagnostic accuracy (Specificity, Sensitivity, Positive predictive value, Negative predictive value).

### 2.1 Comparison with the Reference Test (CLSI Terminology: Trueness/Comparability)

In the current state of the art, free copper, i.e. not bound to ceruloplasmin (Non-Cp copper), is not measured directly, but computed on the basis of the following algorithm (Walsh et al. Ann. Clin. Biochem 2003):

Non-Cp copper=Total copper−0.472×Cp

This procedure determines a percentage of false-negative values equal to 11% in our database. The direct measurement on non-Cp copper, according to the invention, does not determine this error, with an entailed asymmetry of the two distributions. In FIG. 9, the values with the two determinations in the 273 subjects tested are depicted. Linear regression, polynomial and “non-parametric-lowess” (locally weighted scatterplot smoothing) regression analyses indicate that the linear fit is not satisfactory; that inserting the quadratic component into the model significantly enhances adaptation effectiveness (from 0.525 to 0.591, test for R2-change, p<0.001) suggesting the presence of a curvature, and that such curvature can be decomposed, by piecewise regression, into two linear regressions having as critical point value 0 of the calculated non-Cp copper. Since the negative values of the computed non-Cp copper can be considered as procedural errors and are relatively few, accordance between the two measures was carried out exclusively for non-negative values. Considering that it is not two measuring instruments that are being compared, but two detection modes (the standard one, based on the formula binding copper to ceruloplasmin, and the one based on direct measuring according to the present invention), intra-class correlation coefficient was calculated for the evaluation of “consistency” and not of “total accordance”.

Comparison analyses with the reference test indicate that:

Intra-class correlation is equal to 0.75 (95% confidence interval: 0.69-0.80) and no systematic influence exists between the two detection modes (difference test, p=0.959)

| | Paired Differences | | | | | | | |
|---|---|---|---|---|---|---|---|---|
| | | Std. | Std. Error | Interval of the | | | | Sig. (2- |
| | Mean | Deviation | Mean | Lower | Upper | t | df | tailed) |
| Non-Cp copper (measured) - Non-Cp | −.00287 | .86101 | .05523 | −.11167 | .10593 | −.052 | 242 | .959 |
| copper (calculated) | | | | | | | | |

The sample on which the inventors based themselves for defining the reference interval consisted of 147 subjects for which the neurologist had ruled out the presence of cognitive impairment and of past and recent cardio- and cerebrovascular episodes. Average age of control subjects was 49 years (DS=12.8), with 53% of females and 47% of males. Preliminary analysis on the effect of sex and age on non-cp copper indicated that sex has no relevant influence (F(1.140)=0.846; p=0.359, age-squared=0.006) and that age effect of is not significantly different in males and in females (F(1.140)=0.631; p=0.428; age-squared=0.004). Age effect proved statistically significant (F(1.140)=5.114; p=0.035; age-squared=0.035) indicating that 3.5% of non-Cp copper is attributable to age variability. The relationship is substantially linear, with an increase of 0.09 microMol of non-Cp copper for each additional age decade. Then, age-adjusted values were obtained according to the following formula:

Age-adjusted Non-Cp copper=(c4d-0.009*(age-49.05))

The new values were analyzed with the non-parametric CLSI procedure. The upper reference limit (95%) was equal to 1.91 (the related 90% confidence interval was equal to 1.78-2.06).

### 2.2. Discriminating Ability and Numerical Values (Micromols)

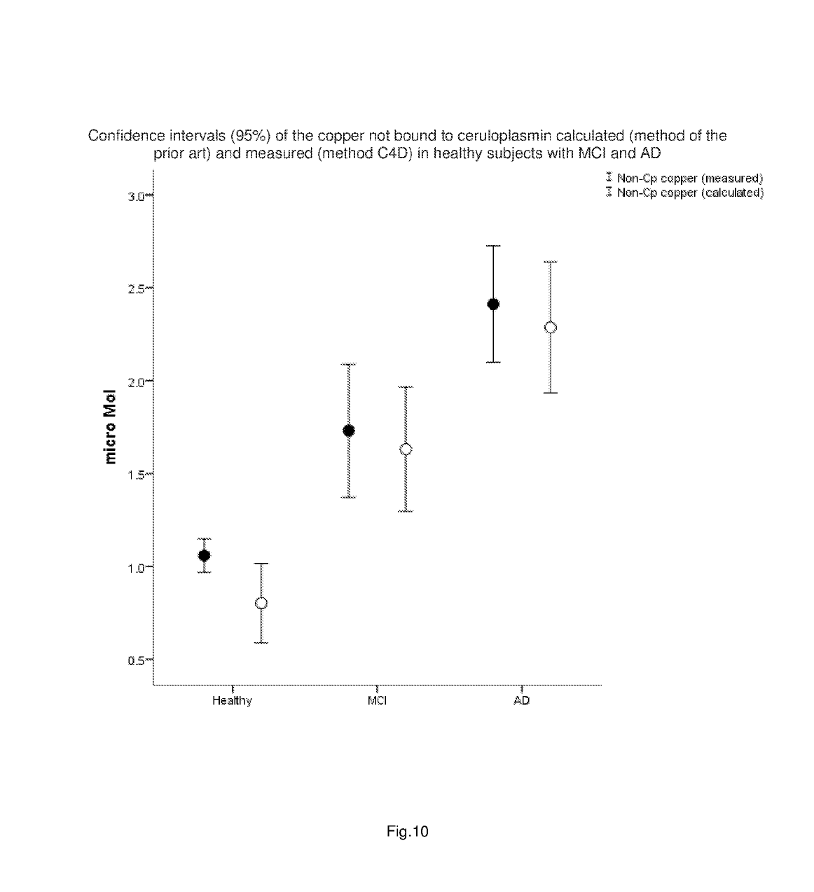

Variance analysis revealed a clear discriminating ability among controls, mild cognitive impairment (MCI) patients and Alzheimer's Disease (AD) patients of both measures (F(2.265)=47.317, p<0.00, age-squared=0.260 for non-Cp copper measured; F(2.265)=32.695, p<0.001, age-squared=0.198 for non-Cp copper calculated according to the method of the state of the art). FIG. 10 depicts the means and confidence intervals of the 3 groups.

Considering only the comparison between controls and patients affected by the target pathology (diagnosis of possible and/or probable Alzheimer's Disease), the ROC curves have shown an accuracy (measured as AUC-Area Under Curve) of 0.761 with non-Cp copper calculated according to the reference test of the state of the art, and of 0.806 with non-Cp copper measured with the method according to the present invention. Such difference proved statistically significant (pairwise ROC comparison, p<0.001). As highlighted in FIG. 11, at a 95% specificity the sensitivity goes from 44% for the determination calculated according to the method of the state of the art to 56% for the determination measured according to the method of the present invention.

### 2.3 Diagnostic Accuracy (Specificity, Sensitivity, Positive Predictive Value, Negative Predictive Value)

At a (95%) specificity set on the basis of the reference limit of the sample of control subjects (1.9), a method sensitivity equal to 48.3% (95% confidence interval:

38%-58%) was detected. The likelihood ratio for positive test (LR+) was 9.94, well above the conventionally accepted cut-off (>5). The likelihood ratio for negative test (LR−) was 0.54, a value not adequate compared to the conventionally accepted cut-off off (<0.2), due to the high percentage of false negatives (AD patients with non-Cp copper values <1.9). To estimate the positive predictive value (PPV) of the test the inventors speculated 3 scenarios, characterized by variable incidences (on the basis of age and of other genetic and clinical conditions).

Hereinafter, some of the results related to the above-described experimenting are summarized in table form. In table 2, the values of diagnostic accuracy attainable with the method of the present invention are summarized. In Table 3.1-3.3, there are reported the values used to process the ROC curves reported in FIG. 11.

TABLE 2

| | | Disease | | | | |
|---|---|---|---|---|---|---|
| | | present | absent | | | |
| TEST | positives | 43 | 7 | 50 | PPV | 65.4% |
| | negatives | 46 | 137 | 183 | NPV | 91% |
| | | 89 | 144 | 233 | | |
| | | 1 | 100 | | | |
| | Prevalence | 16.0% | Accuracy | 77% | LR+ | 9.94 |
| | | | | | LR− | 0.54 |
| | Sensitivity | 48% | 5% | false positive rate | | |
| | false negative rate | 52% | 95% | a priori probability | a priori probability | 0.010 |
| | | | | | a posteriori | 6.143 |
| | | | | | probability | |
| | SE(p) | 0.053 | 0.018 | | PPV | 0.860 |
| | 95% C.I. | 38% | 1% | | | |
| | | 59% | 8% | | | |
| | 0.505 | | | | | |
| | 0.71 | | | | | |
| | 0.365 | | | | | |

TABLE 3
ROC curve coordinates
Table 3.1
Method according to the
present invention (C4D)

| Positive when ≧ | Sensitivity | 1 − Specificity | 1.742 |
|---|---|---|---|
| −1 | 1 | 1 | 1 |
| 0.05 | 0.981 | 0.946 | 1.035 |
| 0.13 | 0.981 | 0.911 | 1.07 |
| 0.18 | 0.981 | 0.893 | 1.088 |
| 0.25 | 0.981 | 0.768 | 1.213 |
| 0.35 | 0.981 | 0.75 | 1.231 |
| 0.45 | 0.981 | 0.732 | 1.249 |
| 0.535 | 0.981 | 0.696 | 1.285 |
| 0.585 | 0.981 | 0.679 | 1.302 |
| 0.65 | 0.981 | 0.625 | 1.356 |
| 0.705 | 0.962 | 0.589 | 1.373 |
| 0.73 | 0.962 | 0.554 | 1.408 |
| 0.775 | 0.962 | 0.536 | 1.426 |
| 0.835 | 0.962 | 0.5 | 1.462 |
| 0.885 | 0.962 | 0.482 | 1.48 |
| 0.92 | 0.962 | 0.464 | 1.498 |
| 0.945 | 0.942 | 0.464 | 1.478 |
| 0.96 | 0.942 | 0.446 | 1.496 |
| 0.985 | 0.942 | 0.429 | 1.513 |
| 1.05 | 0.942 | 0.393 | 1.549 |
| 1.125 | 0.923 | 0.357 | 1.566 |
| 1.175 | 0.923 | 0.339 | 1.584 |
| 1.23 | 0.904 | 0.25 | 1.654 |
| 1.28 | 0.904 | 0.232 | 1.672 |
| 1.35 | 0.904 | 0.196 | 1.708 |
| 1.425 | 0.885 | 0.143 | 1.742 |
| 1.495 | 0.865 | 0.143 | 1.722 |
| 1.545 | 0.865 | 0.125 | 1.74 |
| 1.575 | 0.846 | 0.107 | 1.739 |
| 1.65 | 0.808 | 0.089 | 1.719 |
| 1.71 | 0.788 | 0.089 | 1.699 |
| 1.73 | 0.788 | 0.071 | 1.717 |
| 1.77 | 0.788 | 0.054 | 1.734 |
| 1.825 | 0.75 | 0.054 | 1.696 |
| 1.875 | 0.731 | 0.054 | 1.677 |
| 1.92 | 0.712 | 0.036 | 1.676 |
| 1.97 | 0.692 | 0.036 | 1.656 |
| 2.05 | 0.635 | 0.036 | 1.599 |
| 2.15 | 0.615 | 0.036 | 1.579 |
| 2.285 | 0.577 | 0.036 | 1.541 |
| 2.385 | 0.577 | 0.018 | 1.559 |
| 2.42 | 0.558 | 0.018 | 1.54 |
| 2.47 | 0.538 | 0.018 | 1.52 |
| 2.525 | 0.538 | 0 | 1.538 |
| 2.625 | 0.519 | 0 | 1.519 |
| 2.75 | 0.481 | 0 | 1.481 |
| 2.85 | 0.462 | 0 | 1.462 |
| 2.95 | 0.423 | 0 | 1.423 |
| 3.05 | 0.365 | 0 | 1.365 |
| 3.2 | 0.346 | 0 | 1.346 |
| 3.4 | 0.327 | 0 | 1.327 |
| 3.55 | 0.288 | 0 | 1.288 |
| 3.7 | 0.269 | 0 | 1.269 |
| 3.95 | 0.231 | 0 | 1.231 |
| 4.155 | 0.212 | 0 | 1.212 |
| 4.255 | 0.192 | 0 | 1.192 |
| 4.35 | 0.173 | 0 | 1.173 |
| 4.45 | 0.154 | 0 | 1.154 |
| 4.53 | 0.115 | 0 | 1.115 |
| 4.65 | 0.096 | 0 | 1.096 |
| 5.005 | 0.077 | 0 | 1.077 |
| 5.435 | 0.058 | 0 | 1.058 |
| 5.8 | 0.038 | 0 | 1.038 |
| 6.1 | 0.019 | 0 | 1.019 |
| 7.2 | 0 | 0 | 1 |

Table 3.2
Reference test as described in the state
of the art - Walsh et al. Ann Clin Biochem 2003

| Positive when ≧ | Sensitivity | 1 − Specificity | 1.614 |
|---|---|---|---|
| −9.9412 | 1 | 1 | 1 |
| −7.913 | 1 | 0.982 | 1.018 |
| −6.037652 | 1 | 0.964 | 1.036 |
| −5.074692 | 0.981 | 0.964 | 1.017 |
| −4.4646 | 0.981 | 0.946 | 1.035 |
| −3.751925 | 0.981 | 0.929 | 1.052 |
| −3.374485 | 0.981 | 0.911 | 1.07 |
| −3.0234 | 0.981 | 0.893 | 1.088 |
| −1.81568 | 0.981 | 0.875 | 1.106 |
| −0.747558 | 0.981 | 0.857 | 1.124 |
| −0.644862 | 0.962 | 0.857 | 1.105 |
| −0.431265 | 0.962 | 0.839 | 1.123 |
| −0.243602 | 0.962 | 0.821 | 1.141 |
| −0.139085 | 0.962 | 0.804 | 1.158 |
| 0.020556 | 0.962 | 0.786 | 1.176 |
| 0.115 | 0.962 | 0.768 | 1.194 |
| 0.1854 | 0.962 | 0.75 | 1.212 |
| 0.247374 | 0.962 | 0.732 | 1.23 |
| 0.276974 | 0.962 | 0.714 | 1.248 |
| 0.35 | 0.962 | 0.679 | 1.283 |
| 0.43344 | 0.942 | 0.661 | 1.281 |
| 0.479532 | 0.942 | 0.643 | 1.299 |
| 0.496092 | 0.923 | 0.643 | 1.28 |
| 0.5176 | 0.923 | 0.625 | 1.298 |
| 0.5626 | 0.923 | 0.607 | 1.316 |
| 0.595 | 0.923 | 0.589 | 1.334 |
| 0.64 | 0.923 | 0.571 | 1.352 |
| 0.6884 | 0.923 | 0.554 | 1.369 |
| 0.6984 | 0.923 | 0.536 | 1.387 |
| 0.73 | 0.904 | 0.518 | 1.386 |
| 0.8384 | 0.904 | 0.5 | 1.404 |
| 0.9234 | 0.904 | 0.482 | 1.422 |
| 0.9438 | 0.904 | 0.464 | 1.44 |
| 0.963686 | 0.904 | 0.446 | 1.458 |
| 0.976886 | 0.904 | 0.429 | 1.475 |
| 1.029536 | 0.904 | 0.411 | 1.493 |
| 1.076736 | 0.885 | 0.411 | 1.474 |
| 1.0892 | 0.885 | 0.393 | 1.492 |
| 1.12963 | 0.885 | 0.357 | 1.528 |
| 1.17963 | 0.865 | 0.357 | 1.508 |
| 1.21852 | 0.846 | 0.357 | 1.489 |
| 1.25352 | 0.846 | 0.339 | 1.507 |
| 1.2714 | 0.846 | 0.321 | 1.525 |
| 1.2964 | 0.846 | 0.304 | 1.542 |
| 1.3216 | 0.846 | 0.286 | 1.56 |
| 1.324393 | 0.846 | 0.268 | 1.578 |
| 1.341193 | 0.846 | 0.25 | 1.596 |
| 1.364009 | 0.846 | 0.232 | 1.614 |
| 1.394095 | 0.827 | 0.232 | 1.595 |
| 1.420825 | 0.827 | 0.214 | 1.613 |
| 1.437485 | 0.808 | 0.214 | 1.594 |
| 1.479946 | 0.788 | 0.214 | 1.574 |
| 1.520902 | 0.769 | 0.214 | 1.555 |
| 1.566102 | 0.75 | 0.214 | 1.536 |
| 1.626872 | 0.712 | 0.196 | 1.516 |
| 1.676872 | 0.692 | 0.196 | 1.496 |
| 1.7076 | 0.673 | 0.179 | 1.494 |
| 1.726539 | 0.673 | 0.161 | 1.512 |
| 1.750539 | 0.654 | 0.161 | 1.493 |
| 1.7816 | 0.654 | 0.143 | 1.511 |
| 1.817872 | 0.654 | 0.125 | 1.529 |
| 1.867872 | 0.635 | 0.125 | 1.51 |
| 1.926844 | 0.635 | 0.107 | 1.528 |
| 2.005976 | 0.635 | 0.089 | 1.546 |
| 2.074132 | 0.615 | 0.089 | 1.526 |
| 2.11963 | 0.615 | 0.071 | 1.544 |
| 2.152793 | 0.615 | 0.054 | 1.561 |
| 2.163554 | 0.596 | 0.054 | 1.542 |
| 2.174591 | 0.577 | 0.054 | 1.523 |
| 2.1908 | 0.558 | 0.036 | 1.522 |
| 2.2132 | 0.538 | 0.036 | 1.502 |
| 2.3216 | 0.519 | 0.036 | 1.483 |
| 2.422643 | 0.5 | 0.036 | 1.464 |
| 2.435931 | 0.481 | 0.036 | 1.445 |
| 2.466888 | 0.462 | 0.036 | 1.426 |
| 2.488065 | 0.442 | 0.036 | 1.406 |
| 2.502465 | 0.423 | 0.036 | 1.387 |
| 2.658968 | 0.404 | 0.036 | 1.368 |
| 2.825117 | 0.385 | 0.036 | 1.349 |
| 2.92391 | 0.365 | 0.036 | 1.329 |
| 3.112669 | 0.365 | 0.018 | 1.347 |
| 3.270908 | 0.346 | 0.018 | 1.328 |
| 3.347919 | 0.327 | 0.018 | 1.309 |
| 3.447279 | 0.308 | 0.018 | 1.29 |
| 3.52136 | 0.308 | 0 | 1.308 |
| 3.689376 | 0.288 | 0 | 1.288 |
| 3.931376 | 0.269 | 0 | 1.269 |
| 4.020337 | 0.25 | 0 | 1.25 |
| 4.103137 | 0.231 | 0 | 1.231 |
| 4.1888 | 0.212 | 0 | 1.212 |
| 4.2276 | 0.192 | 0 | 1.192 |
| 4.261576 | 0.173 | 0 | 1.173 |
| 4.344376 | 0.154 | 0 | 1.154 |
| 4.5492 | 0.135 | 0 | 1.135 |
| 5.00515 | 0.115 | 0 | 1.115 |
| 5.380054 | 0.096 | 0 | 1.096 |
| 5.505784 | 0.077 | 0 | 1.077 |
| 5.63511 | 0.058 | 0 | 1.058 |
| 5.839411 | 0.038 | 0 | 1.038 |
| 6.757297 | 0.019 | 0 | 1.019 |
| 8.521832 | 0 | 0 | 1 |

Table 3.3
Copper

| Positive when ≧ | Sensitivity | 1 − Specificity |
|---|---|---|
| −0.1 | 1 | 1 |
| 1.645 | 1 | 0.982 |
| 3.015539 | 1 | 0.964 |
| 4.955539 | 0.981 | 0.964 |
| 6.415 | 0.981 | 0.946 |
| 6.877678 | 0.981 | 0.929 |
| 7.817678 | 0.981 | 0.911 |
| 8.708418 | 0.981 | 0.893 |
| 9.138418 | 0.981 | 0.875 |
| 9.537992 | 0.981 | 0.857 |
| 9.790915 | 0.962 | 0.857 |
| 10.00292 | 0.942 | 0.857 |
| 10.2823 | 0.942 | 0.839 |
| 10.37916 | 0.942 | 0.821 |
| 10.39686 | 0.923 | 0.821 |
| 10.5 | 0.923 | 0.804 |
| 10.73388 | 0.923 | 0.786 |
| 10.87445 | 0.923 | 0.768 |
| 11.04057 | 0.923 | 0.75 |
| 11.4 | 0.923 | 0.732 |
| 11.85 | 0.923 | 0.714 |
| 12.35 | 0.923 | 0.679 |
| 12.62681 | 0.904 | 0.679 |
| 12.65681 | 0.904 | 0.661 |
| 12.68 | 0.904 | 0.643 |
| 12.79977 | 0.904 | 0.625 |
| 12.89977 | 0.885 | 0.625 |
| 12.92462 | 0.865 | 0.607 |
| 12.97462 | 0.865 | 0.589 |
| 13.25 | 0.865 | 0.554 |
| 13.525 | 0.865 | 0.536 |
| 13.57 | 0.865 | 0.518 |
| 13.595 | 0.865 | 0.5 |
| 13.60834 | 0.865 | 0.482 |
| 13.65834 | 0.865 | 0.464 |
| 13.745 | 0.846 | 0.446 |
| 13.845 | 0.846 | 0.429 |
| 13.90649 | 0.846 | 0.411 |
| 13.92627 | 0.827 | 0.411 |
| 14.01978 | 0.788 | 0.411 |
| 14.10721 | 0.75 | 0.393 |
| 14.15721 | 0.731 | 0.393 |
| 14.24428 | 0.731 | 0.321 |
| 14.29428 | 0.712 | 0.321 |
| 14.33914 | 0.712 | 0.304 |
| 14.40016 | 0.712 | 0.286 |
| 14.42448 | 0.712 | 0.268 |
| 14.46347 | 0.692 | 0.268 |
| 14.53 | 0.673 | 0.268 |
| 14.68782 | 0.673 | 0.25 |
| 14.85782 | 0.673 | 0.232 |
| 14.955 | 0.654 | 0.232 |
| 15.10698 | 0.654 | 0.214 |
| 15.25198 | 0.635 | 0.214 |
| 15.328 | 0.596 | 0.179 |
| 15.368 | 0.577 | 0.179 |
| 15.39 | 0.577 | 0.161 |
| 15.416 | 0.558 | 0.161 |
| 15.466 | 0.538 | 0.161 |
| 15.50861 | 0.538 | 0.143 |
| 15.55861 | 0.519 | 0.143 |
| 15.65 | 0.5 | 0.143 |
| 15.75 | 0.5 | 0.125 |
| 15.80859 | 0.481 | 0.107 |
| 15.85553 | 0.462 | 0.107 |
| 15.89943 | 0.442 | 0.107 |
| 15.95249 | 0.423 | 0.107 |
| 16.05 | 0.404 | 0.107 |
| 16.10725 | 0.346 | 0.107 |
| 16.20725 | 0.346 | 0.089 |
| 16.45 | 0.346 | 0.071 |
| 16.65 | 0.327 | 0.071 |
| 16.78 | 0.327 | 0.054 |
| 16.88503 | 0.327 | 0.036 |
| 17.04051 | 0.308 | 0.036 |
| 17.39034 | 0.308 | 0.018 |
| 17.64841 | 0.288 | 0.018 |
| 17.69354 | 0.269 | 0.018 |
| 17.85 | 0.25 | 0.018 |
| 18.0378 | 0.25 | 0 |
| 18.18962 | 0.231 | 0 |
| 18.70182 | 0.212 | 0 |
| 19.51686 | 0.192 | 0 |
| 20.32224 | 0.173 | 0 |
| 21.08389 | 0.154 | 0 |
| 21.4724 | 0.135 | 0 |
| 21.64389 | 0.115 | 0 |
| 21.85 | 0.096 | 0 |
| 21.92616 | 0.077 | 0 |
| 22.2606 | 0.058 | 0 |
| 23.42192 | 0.038 | 0 |
| 24.38999 | 0.019 | 0 |
| 25.50501 | 0 | 0 |

1. An in vitro method for determining the concentration of free copper in a serum sample comprising the following steps:
(a) loading said serum sample on a resin for solid phase extraction to obtain a bonded fraction and an eluted fraction comprising free copper; and
(b) determining the concentration of free copper in the fraction eluted in step (a) using a coumarin fluorescent probe.

2. The method according to claim 1 wherein said resin is a polyolefin.

3. The method according to claim 1 wherein said resin is ultra-high molecular weight polyethylene.

4. The method according to claim 1 wherein said step (a) uses a physiological solution as mobile phase.

5. The method according to claim 1 wherein said coumarin fluorescent probe is used in a concentration range between 0.1 and 10 μM.

6. The method according to claim 1 wherein said coumarin fluorescent probe is used in a solution of HEPES:DMSO.

7. The method according to claim 1 wherein said step (b) comprises a step of preparation of a calibration curve.

8. The method according to claim 1 wherein the determination of the concentration of free copper in said step (b) is obtained by reading the fluorescence of said coumarin probe at a wavelength of excitation (Aₑₓ) of 430 nm and a wavelength of absorption (Aₑₘ) of 490 nm.

9. The method according to claim 1 wherein said coumarin fluorescent probe is selected from the group consisting of compounds having the following general structural formula: 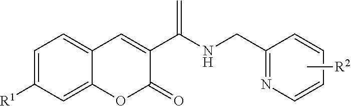
wherein
R¹ is N[(CH₂)nCH₃]₂ with n from 0 to 5;
R² is H, F, CI, Br, NO₂, OCH₃, or cyclohexyl.

10. The method according to claim 1 wherein said coumarin fluorescent probe is selected from the compounds having the following general structural formula:

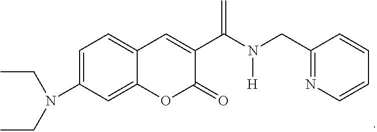 [SMILES: C=C(NCc1ccccn1)c1cc2ccc(N(CC)CC)cc2oc1=O]

11. The method according to claim 1 for the diagnosis of Alzheimer's disease in a patient comprising a step c) of comparing the value determined in step b) with a threshold value (cut-off), wherein a higher concentration of free copper confirms the clinical diagnosis of Alzheimer's disease.

12. The method according to claim 11 wherein said threshold value is between 0.5 and 50 μM.

13. The method according to claim 11 for the prognosis of Alzheimer's disease in a patient in which the steps a) and b) of the method are repeated on serum samples collected from said patient at subsequent time-points and the progression in time of the concentration of free copper in these samples is evaluated.

14. The method according to claim 1 for the evaluation of the predisposition to conversion from a state of mild cognitive impairment (MCI) to Alzheimer's disease in a patient suffering from mild cognitive impairment comprising a step c) of comparing the value determined in step b) with a threshold value (cut-off), wherein a higher concentration of copper points out the conversion from mild cognitive impairment to Alzheimer's disease.

15. The method according to claim 14 wherein said threshold value is between 0.5 and 3 μM.

16. A kit for the detection of free copper in serum comprising one or more means for performing a chromatographic extraction on a solid phase and one or more fluorescent coumarin probes.

17. The kit according to claim 16 wherein said means comprise as solid phase ultra-high molecular weight polyethylene.

18. The kit according to claim 16, wherein said coumarin fluorescent probe is selected from the group consisting of compounds having the following general structural formula: 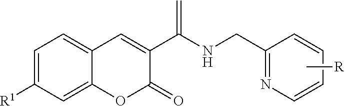
wherein
R¹ is N[(CH₂)nCH₃]₂ with n from 0 to 5;
R² is H, F, CI, Br, NO₂, OCH₃, or cyclohexyl.
